(19) United States
(12) Patent Application Publication
(10) Pub. No.: US 2025/0169746 A1
(43) Pub. Date: May 29, 2025

(54) INTELLIGENT SYSTEM AND USE THEREOF

(71) Applicant: National Taiwan University, Taipei (TW)

(72) Inventors: WEI-LI HSU, Taipei (TW); BIING-CHWEN CHANG, Taipei (TW)

(21) Appl. No.: 18/962,330

(22) Filed: Nov. 27, 2024

Related U.S. Application Data
(60) Provisional application No. 63/603,366, filed on Nov. 28, 2023.

(51) Int. Cl. A61B 5/00; A61B 5/11; G16H 20/30

(52) U.S. Cl. CPC A61B 5/4585; A61B 5/1121; A61B 5/4836; A61B 5/6828; A61B 5/7246; A61B 5/7264; G16H 20/30; A61B 2560/0431

(57) ABSTRACT

The present application provides an intelligent system, including: at least one sensor configured to receive at least one information of current knee movement, which generates sensing inputs; a controller configured to receive and compute the sensing inputs, and configured to predict knee motion to generate a predicted knee movement; an analyzer configured to compare the predicted knee movement and an ideal knee movement; and at least one functional unit configured to provide outputs of stimulations according to comparison results from the analyzer; wherein the outputs of stimulations includes outputs of stimulations after adjustment or without adjustment. Besides, the present application also provides a use of an intelligent system, wherein the use includes purposes of health-care, rehabilitation, preventive medicine or medical use.

### CROSS REFERENCE

This application claims priority to, and the benefit of, U.S. Provisional Application No. 63/603,366, filed on Nov. 28, 2023, the content thereof is incorporated by reference herein.

### FIELD OF THE INVENTION

The present invention relates to an intelligent system, especially an intelligent physiological sensing system, use thereof, and to methods of using the intelligent system thereof.

### BACKGROUND OF THE INVENTION

Nowadays, subject with knee arthritis or limited knee joint mobility often experience symptoms such as chronic pain, muscle weakness, difficulty with daily activities, reduced mobility, balance problems, and walking impairments, which can greatly affect their quality of life and elevate the risk of disability.

However, most knee joint assistive devices currently available on the market generally provide single-function solutions or pre-fined values of stimulations, which lacks the ability to deliver adaptive or personalized stimulations during dynamic or static activities. So as to, individuals with degenerative arthritis often need to visit the hospital frequently for high-frequency rehabilitation or treatment, which causes great inconvenience for those with limited mobility, and reduces adherence to rehabilitation therapy.

Accordingly, the intelligent systems providing real-time feedback and joint assistive devices comprising thereof have barely been discussed. Thus, development of a home-use, portable joint assistive devices which enhances a subject's willingness and frequency to engage in exercise training at home, is urgently needed from academic to clinical research.

### SUMMARY OF THE INVENTION

The invention is based on the discovery that an intelligent system, which exhibits unexpected real-time feedback loop where machine learning refines outputs of stimulations, providing more accurate and responsive assistance based on complex joint movement patterns properties.

Accordingly, the present invention provides an intelligent system, including: at least one sensor configured to receive at least one information of current knee movement, which generates sensing inputs; a controller configured to receive and compute the sensing inputs, and configured to predict knee motion to generate a predicted knee movement; an analyzer configured to compare the predicted knee movement and an ideal knee movement; and at least one functional unit configured to provide outputs of stimulations according to comparison results from the analyzer; wherein the outputs of stimulations includes outputs of stimulations after adjustment or without adjustment.

Further, the intelligent system provided herein is configured to be wearable and portable.

Further, the sensor provided herein includes an accelerometer, a gyroscope, a magnetometer, a barometer, a flex sensor, a potentiometer or an electromyography.

Further, the information of current knee movement provided herein includes knee angle, knee velocity, angular velocity of knee, angular acceleration of knee or muscle activity in spatiotemporal patterns.

Further, the controller provided herein includes a microcontroller.

Further, the predicted knee movement provided herein is provided through Deep Neural Network (DNN) model, a Convolutional Neural Network (CNN) model, Recurrent Neural Network (RNN) model or combination thereof.

Further, the ideal knee movement provided herein includes ideal knee angle, knee velocity, angular velocity of knee, angular acceleration of knee or muscle activity in spatiotemporal patterns.

Further, the analyzer provided herein is configured to compare the predicted knee movement and the ideal knee movement, which generates an error rate.

Further, the error rate provided herein meets a formula as follows:

$$error rate = \frac{current knee movement - ideal knee movement}{ideal knee movement} .$$

Further, the intelligent system provided herein further includes at least one parameter convertor, which is configured to adjust parameters regulating the functional unit according to the error rate; and wherein the parameter convertor is communicatively connected between the analyzer and the functional unit.

Further, the intelligent system provided herein further includes at least one parameter convertor, which is configured to adjust parameters regulating the functional unit according to comparison results from the analyzer; and wherein the parameter convertor is communicatively connected between the analyzer and the functional unit.

Further, the parameter convertor provided herein includes a vibration, infrared stimulation, laser, thermal or electrical stimulation parameter convertor.

Further, the parameter convertor provided herein is configured to adjust at least one parameter of amplitude, frequency, frequency range, time duration, action sites, spatiotemporal pattern, infrared wavelength and temperature.

Further, the parameter convertor provided herein is configured to adjust parameters through Deep Neural Network (DNN) model, a Convolutional Neural Network (CNN) model, Recurrent Neural Network (RNN) model or combination thereof.

Further, the functional unit provided herein includes a vibration motor, an infrared instrument, a laser instrument, a heating instrument, a cooling instrument or an electrical stimulation instrument.

In another aspect, the present invention provides a use of an intelligent system, wherein the use includes purposes of health-care, rehabilitation, preventive medicine or medical use.

Accordingly, the intelligent system of the present invention can exhibit real-time sensing, motion identification, motion prediction, comparison with the predicted movement and ideal model, and provides unexpected real-time feedback loop wherein deep learning model or machine learning model continuously refines parameter used for regulating the functional units. As such, the functional units provide improved, adaptive and responsive stimulation during dynamic and static movements, thereby allowing individuals adherence to rehabilitation therapy and increasing the efficiency of health care, rehabilitation or treatment.

### BRIEF DESCRIPTION OF THE DRAWINGS

In order to make the above and other objects, features, advantages and embodiments of the present invention more obvious and understandable, the drawings are described as follows:

FIG. 1 illustrates a diagram embodiment of intelligent system;

FIG. 2 illustrates another diagram embodiment of intelligent system;

FIG. 3 illustrates a diagram embodiment of flow chart using the intelligent system thereof;

FIG. 4 illustrates another diagram embodiment of flow chart using another intelligent system thereof;

FIG. 5 illustrates a diagram embodiment of flow chart using the intelligent system thereof;

FIG. 6 illustrates a diagram embodiment of data of the predicted knee movement and an ideal knee movement, and comparison results thereof; and

FIG. 7 illustrates a diagram embodiment of a subject wearing the assistive device comprising the intelligent system.

### DETAILED DESCRIPTION OF THE INVENTION

### Definition

The terms used in this specification are generally within the scope of the present invention and the specific context of each term has its usual meaning in related fields. The specific terms used to describe the present invention in this specification will be described below or elsewhere in this specification, so as to help people in the industry understand the relevant description of the present invention. The same term has the same scope and meaning in the same context. In addition, there are more than one way to express the same thing; therefore, the terms discussed in this article may be replaced by alternative terms and synonyms, and whether a term is specified or discussed in this article does not have any special meaning. This article provides synonyms for certain terms, but the use of one or more synonyms does not mean that other synonyms are excluded.

As used herein, unless the context clearly indicates otherwise, “a” and “the” can also be interpreted as plural. Furthermore, titles and subtitles may be attached to the description for easy reading, but these titles do not affect the scope of the present invention.

As used herein, the term “intelligent system” may also refer to an intelligent physiological sensing system, which provides services including, but not limited to, intelligent sensing, motion identification, motion prediction, comparison with the predicted movement and ideal model, outputs of stimulation, outputs of stimulation after adjustment or combination thereof, based on artificially intelligent.

The present invention provides an intelligent system, including: at least one sensor configured to receive at least one information of current knee movement, which generates sensing inputs; a controller configured to receive and compute the sensing inputs, and configured to predict knee motion to generate a predicted knee movement; an analyzer configured to compare the predicted knee movement and an ideal knee movement; and at least one functional unit configured to provide outputs of stimulations according to comparison results from the analyzer; wherein the outputs of stimulations includes outputs of stimulations after adjustment or without adjustment.

In some embodiments, the intelligent system provided herein is configured to be wearable and portable.

In some embodiments, the sensor provided herein is configured to autonomously and continuously collect sensing data from a subject, wherein the subject includes, but not limited to, human beings, pet or wild animals.

In some embodiments, the current movement has spatial correlation of events. For example, in motion or physiological monitoring, sensors placed on different parts of the body (such as the knee, hip, etc.) may measure data that exhibit spatial correlation, as the movements of these areas occur in coordination with each other.

In some embodiments, the current movement includes, but not limited to, dynamic or static movement, wherein the dynamic movement includes, but not limited to, climbing stairs, walking, laying with knee flex, running or jumping.

In some embodiments, the target of the sensor configured to receive the information of current movement includes, but not limited to, knee, knee joint, ankle joint, shoulder joint, cervical joint, elbow joint, wrist joint, hip joint, spinal joints, finger joints, toe joints, sacroiliac joint, sternoclavicular joint, acromioclavicular joint, lateral and medial collateral ligaments, muscle movement, lumbar spine or combination thereof.

In some embodiments, the sensor provided herein includes, but not limited to, inertial measurement unit (IMU) sensor.

In some embodiments, the sensor provided herein includes, but not limited to, accelerometer, gyroscope, magnetometer, barometer, flex sensor, potentiometer or electromyography, as shown in Table 1.

TABLE 1

| Measurement | Sensor | Example Use |
|---|---|---|
| Joint angle, | Accelerometer (g (m/s²)) | Collect the data |
| angular | Gyroscope (rad/s) | from sensors on the |
| velocity, | Magnetometer (tesla) | thigh and shank. |
| angular | Potentiometer (degree) | Compute the |
| acceleration | Flex sensor (degree) | difference between |
| | | the two segments. |
| Range of | Accelerometer (g (m/s²)) | Compute the angle |
| motion | Gyroscope (rad/s) | based on joint |
| | Magnetometer (tesla) | angle |
| | Potentiometer (degree) | measurement. |
| | Flex sensor (degree) | |
| Motion | Accelerometer (g (m/s²)) | Detect segment |
| detection | Gyroscope (rad/s) | angle and elevation |
| | Magnetometer (tesla) | to classify |
| | Barometer (Pa) | movements. |
| Muscle activity | Electromyography | Measure muscle |
| | (Voltage) | activation level |

In some embodiments, to detect joint angle, angular velocity, angular acceleration, sensors such as accelerometers, gyroscopes, magnetometers, potentiometers, flex sensors or combination thereof can be used for collecting data from sensors on the thigh and shank, or computing the difference between the two segments.

In some embodiments, to detect range of motion, sensors such as accelerometers, gyroscopes, magnetometers, potentiometers, flex sensors or combination thereof can be used for computing joint angle based on joint angle measurements.

In some embodiments, when motion detection is required, sensors such as accelerometers, gyroscopes, magnetometers, barometers or combination thereof can be used for detecting segment angle and elevating to classify movements.

In some embodiments, to detect muscle activity, sensors such as electromyography can be used for measuring muscle activation level.

More specifically, the information of current knee movement provided herein includes, but not limited to, knee angle, knee velocity, angular velocity of knee, angular acceleration of knee or muscle activity in spatiotemporal patterns.

In some embodiments, the controller provided herein is communicatively connected to the sensor.

In some embodiments, the controller is configured to receive and compute the sensing inputs, and to identify motion and predict motion to generate a predicted movement.

In some embodiments, the controller provided herein includes, but not limited to, microchip, microcontroller (IMU), microprocessor, system on chip (SoC), embedded system, programmable logic controller (PLC), development board or single-board computer (SBC). In some embodiments, the SoC is configured for data acquisition and processing of an inertial measurement unit (IMU).

In some embodiments, the controller provided herein is able to communicatively connect to remote computer via the communication network.

In some embodiments, the controller provided herein is configured to communicatively connect to database, wherein the database is configured to store the information used in motion analyzing, motion identification, motion prediction or the combination thereof.

In some embodiments, the predicted knee movement provided herein is provided through mechanism learning model or deep learning model, preferably deep learning model. In some embodiments, the predicted knee movement provided herein is provided based on the information of current movement corresponding to the model.

In some embodiments, the predicted knee movement provided herein is provided through Deep Neural Network (DNN) model, a Convolutional Neural Network (CNN) model, Recurrent Neural Network (RNN) model or combination thereof.

In some embodiments, the RNN model provided herein further comprises, but not limited to, Long-Short Term Memory (LSTM) model.

In some embodiments, the analyzer provided herein is configured to compare the predicted knee movement and the ideal knee movement, and to generate an error rate.

In some embodiments, the ideal knee movement provide herein is based on human anthropometric data of more than ten people, which is obtained through supervised learning. In some embodiments, as subject numbers of detection-related data increases, the ideal knee movement provide herein is obtained through continuously updated model size and accuracy.

In some embodiments, the ideal knee movement provided herein includes, but not limited to, ideal knee angle, knee velocity, angular velocity of knee, angular acceleration of knee or muscle activity in spatiotemporal patterns.

In some embodiments, the error rate provided herein meets a formula as follows:

$$error rate = \frac{current knee movement - ideal knee movement}{ideal knee movement} .$$

In some embodiments, the analyzer provided herein includes, but not limited to, microchip, microcontroller (IMU), microprocessor, system on chip (SoC), embedded system, programmable logic controller (PLC), development board or single-board computer (SBC). In some embodiments, the SoC is configured to data acquisition and processing of an inertial measurement unit (IMU).

In some embodiments, the controller and the analyzer are the USB logic analyzer 24 MHz 8 CH microcontrollers.

In some embodiments, the intelligent system provided herein further includes at least one parameter convertor, which is configured to adjust parameters regulating the functional unit according to the error rate; and wherein the parameter convertor is communicatively connected between the analyzer and the functional unit.

In some embodiments, the intelligent system provided herein further includes at least one parameter convertor, which is configured to adjust parameters regulating the functional unit according to comparison results from the analyzer; and wherein the parameter convertor is communicatively connected between the analyzer and the functional unit.

In some embodiments, the parameter convertor provided herein includes, but not limited to, a vibration, infrared stimulation, laser, thermal or electrical stimulation parameter convertor.

In some embodiments, the parameter convertor provided herein is configured to adjust at least one parameter of amplitude, frequency, frequency range, time duration, action sites, spatiotemporal pattern, infrared wavelength and temperature.

In some embodiments, the parameter convertor provided herein is configured to adjust parameters through Deep Learning model or Mechanism Learning model, preferably Deep Learning model.

In some embodiments, the parameter convertor provided herein is configured to adjust parameters through Deep Learning model, which includes, but not limited to, Deep Neural Network (DNN) model, a Convolutional Neural Network (CNN) model, Recurrent Neural Network (RNN) model or combination thereof.

In some embodiments, the RNN model provided herein further comprises, but not limited to, Long-Short Term Memory (LSTM) model.

In some embodiments, the LSTM model is used to detect sensing inputs related to movements. The structure of the LSTM model includes layers as follows: a least one of input layer, encoder layers, at least one bottleneck, decoder layers and at least one of output layer. Wherein the input layer is configured to input time-series sensor data; wherein the encoder layers are configured to progressively reduce the data to a compressed latent representation; wherein the bottleneck is configured to encode representation of the input data; wherein the decoder Layers are configured to reconstruct the input from the latent space; and wherein the output layer is configured to output reconstructed data.

In some embodiments, the functional unit provided herein includes, but not limited to, a vibration motor, an infrared instrument, a laser instrument, a heating instrument, a cooling instrument or an electrical stimulation instrument.

In another aspect, the present invention provides a use of an intelligent system, wherein the use includes purposes of health-care, rehabilitation, preventive medicine or medical use. More specifically, the rehabilitation provided herein includes rehabilitation exercise training.

In some embodiments, the medical use provided herein includes, but not limited to, development of assistive devices for degenerative joints.

In some embodiments, the intelligent system provided herein can be used in an assistive device, such as multifunctional artificial intelligence knee brace.

In some embodiments, the sensors and deep learning model are configured to identify movement even if the user performs the same joint angle pattern.

### EXAMPLES

In this section, the contents of the present invention will be described in detail through the following examples. These examples are for illustration only, and those skilled in the art can easily think of various modifications and changes. As such, various embodiments of the present invention will be described in detail below, while the invention is not limited to said various embodiments listed in this specification.

### Example 1

### Intelligent System 10a

Patients, undergoing knee joint rehabilitation, use the intelligent system 10a as follows:

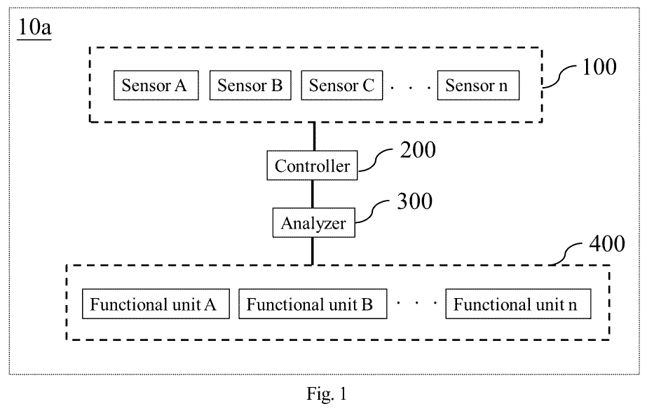

As shown in FIG. 1, the intelligent system 10a comprises: three sensors 100 (Sensor A, Sensor B and Sensor C are all electromyography) configured to receive information of current knee movement (such as muscle activity), which generates sensing inputs; a controller 200 (such as IMU) configured to receive and compute the sensing inputs, and to predict knee motion to generate a predicted knee movement; an analyzer 300 (such as IMU) configured to compare the predicted knee movement and an ideal knee movement; and two functional units 400 (functional unit A and functional unit B are both electrical stimulation instruments) configured to provide outputs of stimulations according to comparison results from the analyzer 300; wherein the outputs of stimulations include outputs of stimulations after adjustment or without adjustment.

More specifically, a patient with knee arthritis wears the assistive device comprising intelligent system 10a during exercise training. The intelligent system 10a includes three electromyography (EMG) sensors (Sensor A, Sensor B, and Sensor C 100), which are attached to the patient's thigh and calf muscles to measure muscle activity during knee movement. These sensors 100 collect muscle activity data in real-time and transmit it to the controller 200 (such as IMU). Once the controller 200 receives the sensor data, it calculates the current knee movement status and uses algorithms to generate a predicted movement. The analyzer 300 then compares the predicted knee movement with the ideal knee movement. According to comparison results from the analyzer 300, the functional units 400 output corresponding electrical stimulation to assist and recovery muscle activity during knee movement based on the complex joint movement pattern.

### Example 2

### Intelligent System 10b

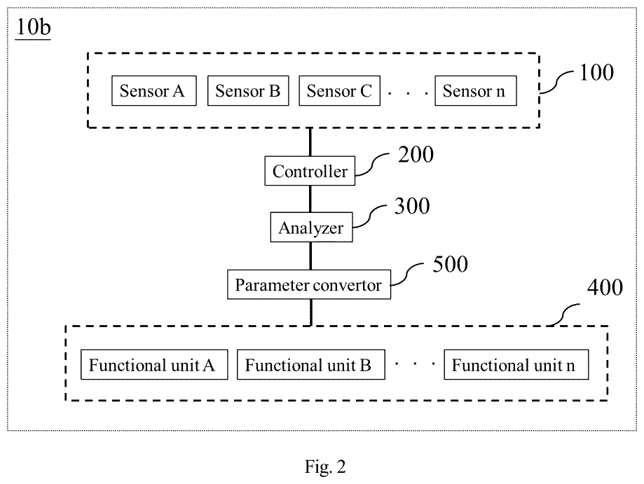

As shown in FIG. 1 and FIG. 2, the difference between the intelligent system 10a and intelligent system 10b is that the intelligent system 10b further comprises a parameter convertor 500 (such as electrical stimulation parameter convertor), wherein the parameter convertor 500 is configured to output parameters used for regulating the functional unit 400, such as regulating intensity, amplitude or frequency of electrical stimulation, according to sensing input and comparison results from the analyzer 300; and wherein the parameter convertor 500 is communicatively connected between the analyzer 300 and the functional unit 400.

When the predicted knee movement matches with the ideal knee movement, for example: the predicted muscle activation level meets the ideal muscle activity level, the parameter convertor 500 outputs parameters used for regulating functional units 400 based on corresponding sensing input.

Therefore, the intelligent system 10b, by incorporating a parameter converter 500, exhibits more precise parameter conversion for regulating the functional units 400 and improved electrical stimulation based on sensing input and movement comparison results, thereby allowing more effective supports for the patient's recovery treatment.

### Example 3

### Flow Chart Using the Intelligent System Thereof

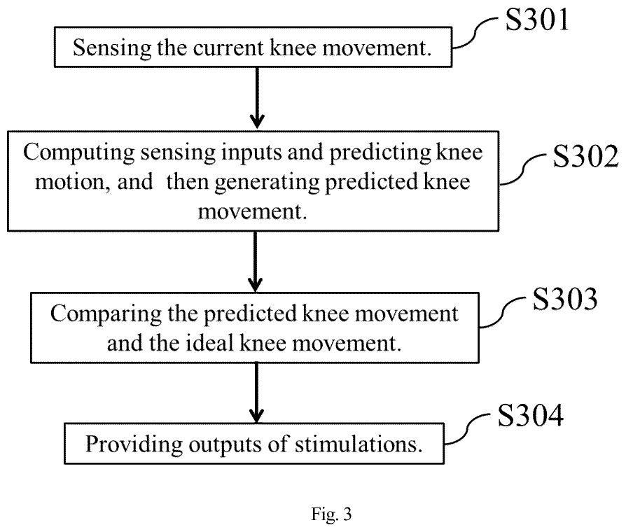

As shown in FIG. 3, the method of implementing the intelligent system comprises:
S301: Sensing the current knee movement;
S302: Computing sensing inputs and predicting knee motion, and then generating predicted knee movement;
S303: Comparing the predicted knee movement and the ideal knee movement; and
S304: Providing outputs of stimulations.
   In step S301, data including muscle activity and knee angle during the patient's knee movement are collected in real time through sensors 100 configured around the knee. These sensing inputs reflect the information of knee movement in time-series.
   In step S302, based on the collected sensing inputs, the controller 200 performs calculations and predicts the movement trajectory of the patient's knee, which generates a predicted knee movement.
   In step S303, the analyzer 300 compares the knee angle of predicted knee movement with ideal knee movement to determine the difference between them.
   In step S304, according the comparison results, the functional unit 400 generates outputs of the electrical stimulation to provide appropriate electrical stimulation to the patient to assist in knee movement and recovery.

### Example 4

### Flow Chart Using the Intelligent System Thereof

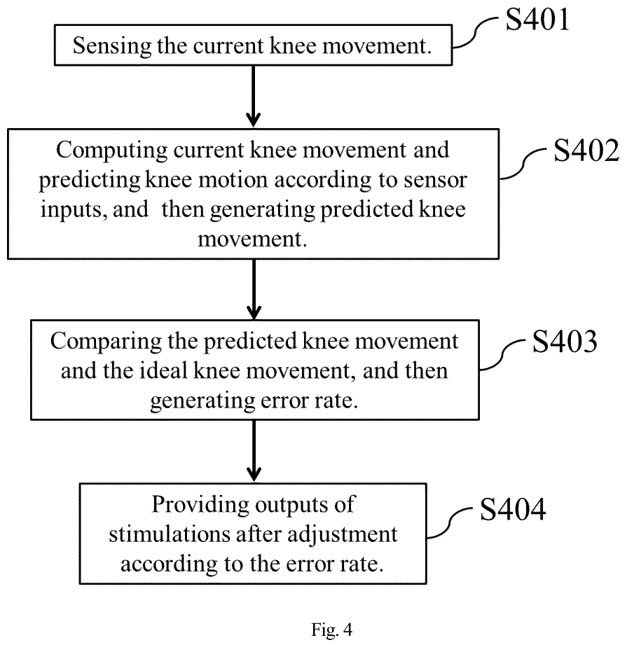

As shown in FIG. 4, the method of implementing the intelligent system comprises:
S401: Sensing the current knee movement;
S402: Computing sensing inputs and predicting knee motion, and then generating predicted knee movement;
S403: Comparing the predicted knee movement and the ideal knee movement, and then generating error rate; and
S404: Providing outputs of stimulations after adjustment according to the error rate.

The steps S401 and S402 are substantially the same as the content described in Example 3.

In step S403, the controller 200 compares the predicted knee movement to ideal knee movement (such as knee angle and muscle activity), and calculates the difference between them. The analyzer 300 generates an error rate according to the differences between the predicted knee movement and ideal knee movement. For example, in the comparison of knee joint flexion angle, the ideal knee movement is set to be 100 (namely the knee angle is 90 degree), and the current knee movement is 25% (namely the knee angle is 22.5 degree), so the error rate is 75%. The results of the error rate show that the current knee joint flexion angle has merely reached 25%.

In step S404, the functional unit provides outputs of stimulations based on the adjusted parameters according to the error rate, and provides improved electrical stimulation (e.g., increasing the muscle activation to evaluate the knee angle) to the patient to achieve preferred ideal knee motion state, thereby promoting recovery and aid in rehabilitation.

### Example 5

### The Intelligent System Provides Stimulation Adjustment Loop

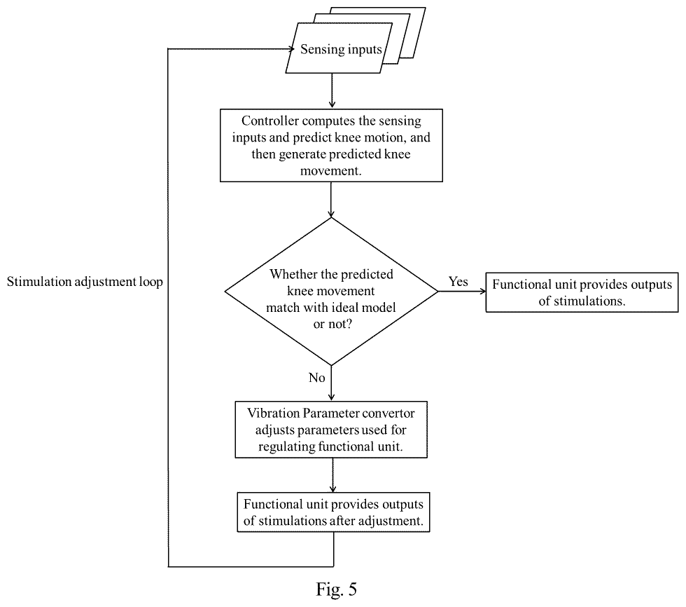

As shown in FIG. 5, it is a flow chart using the intelligent system of the present invention, wherein the intelligent system provides stimulation adjustment loop, which is preferably a closed-loop control to adjust the output of stimulation in real time. Taking the vibration stimulation for example, the related parameters like amplitude and frequency are both proportional to the joint angle and angular velocity, thereby it can be ensured that the vibration motor responds more aggressively to fast movements.

More specifically, when the predicted knee movement (such as predicted knee angle and predicted muscle activation (namely the current knee angle and muscle activation respectively) are different from the ideal model, the error rate e=(current-ideal)/ideal is computed. The parameters (like frequency and the amplitude) for regulating the vibration motor will be adjusted based on the error rate. After the functional unit provides outputs of stimulations with adjustment, the sensors collect the information of current knee movements again. The stimulation adjustment loop will continue until the comparison result between the predicted knee movements and the ideal one becomes consistent, which forms a closed-loop control to adjust the outputs of stimulation in real time.

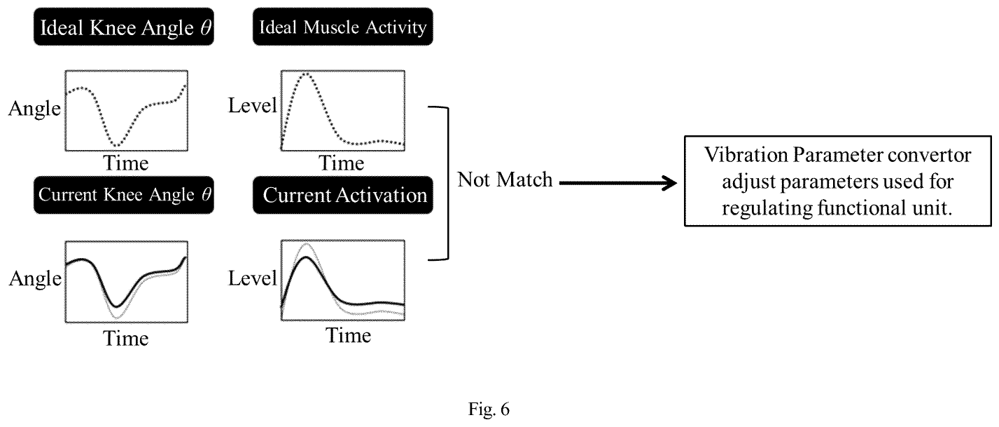

Besides, as shown in FIG. 6, the data of the predicted knee movement, an ideal knee movement, and comparison results thereof are presented. When the current movement of the knee joint does not match with the ideal movement, the parameter converter will adjust the parameter for regulating the functional unit. After the parameters are adjusted, the functional unit outputs improved electrical stimulation based on the comparison results to accurately assist the muscles activation.

### Example 6

### Subject Wears the Assistive Device Comprising the Intelligent System 10c

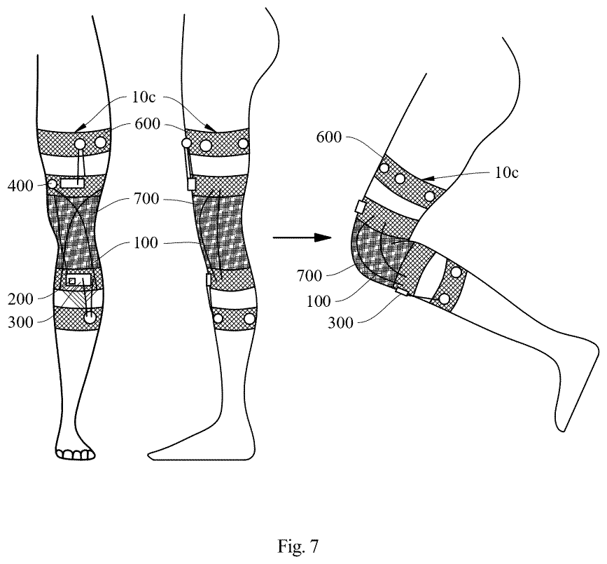

As shown in FIG. 7, the assistive device is a multifunctional vibrating knee brace known as the AI-knee brace, wherein intelligent system 10c includes a sensor 100 (flex sensor), a controller 200 (microcontroller), an analyzer 300 (IMU), a functional unit 400 (vibration motor), secure brackets 600 and soft fabric 700. As the posture of left foot wearing the assistive device changes from standing posture to knee flexing, the flex sensor 100 senses the information of knee movements, such as knee angle and velocity in time-series pattern, and transmits it to the microcontroller 200. The vibration motor 400 outputs the corresponding stimulation in real time, such as vibration, based on the parameters generated from microcontroller 200 and IMU 300. In this way, the assistive device providing personalized vibrations can assist in recovery of knee joint degeneration and relieve pain.

The assistive device provided herein provides vibration therapy to relieve pain and improve local blood circulation during both dynamic and static activities. In addition, the brace is equipped with a flex sensor (sensor 100) and operated using artificial intelligence (AI), which allows it to identify functional movement deficiencies during at-home use or rehabilitation exercises. For instance, the intelligent system can evaluate specific functional movements by creating a data repository (such as knee joint motion and strength in a healthy individual performing a deep squat). Later, when the device is used to track knee joint motion and strength in individuals with knee arthritis or compromised knee mobility, the intelligent system compares the current measurements with the pre-established database. The feedback is then displayed on the screen, informing users of the degree of their movement deficiencies (e.g., a knee joint flexion angle of only 75%). This embodiment aims to offer users in-time and accurate healthcare or professional scientifically reliable reference data.

In summary, the intelligent system of the present invention can exhibit real-time sensing, motion identification, motion prediction, comparison with the predicted movement and ideal model, and provides unexpected real-time feedback loop where deep learning model or machine learning model continuously refines parameter used for regulating the functional units. As such, the functional units can provide improved, adaptive and/or responsive stimulation during dynamic and static movements, thereby allowing individuals adherence to rehabilitation therapy and increasing the efficiency of health care, rehabilitation or treatment.

The specific embodiments of the present invention have been disclosed, but it is not intended to limit the present invention. Those with ordinary knowledge in the technical field to which the present invention belongs are capable of understanding. And in the case of deviating from the principle and spirit of the present invention, various changes and modifications can be made to it, so the scope of protection of the present invention should be based on those defined in the scope of the accompanying patent application.

What is claimed is:

1. An intelligent system, including:
at least one sensor configured to receive at least one information of current knee movement, which generates sensing inputs;
a controller configured to receive and compute the sensing inputs, and configured to predict knee motion to generate a predicted knee movement;
an analyzer configured to compare the predicted knee movement and an ideal knee movement; and
at least one functional unit configured to provide outputs of stimulations according to comparison results from the analyzer;
wherein the outputs of stimulations includes outputs of stimulations after adjustment or without adjustment.

2. The intelligent system according to claim 1, wherein the intelligent system is configured to be wearable and portable.

3. The intelligent system according to claim 1, wherein the sensor includes an accelerometer, a gyroscope, a magnetometer, a barometer, a flex sensor, a potentiometer or an electromyography.

4. The intelligent system according to claim 1, wherein the information of current knee movement includes knee angle, knee velocity, angular velocity of knee, angular acceleration of knee or muscle activity in spatiotemporal patterns.

5. The intelligent system according to claim 1, wherein the controller includes a microcontroller.

6. The intelligent system according to claim 1, wherein the predicted knee movement is provided through Deep Neural Network (DNN) model, a Convolutional Neural Network (CNN) model, Recurrent Neural Network (RNN) model or combination thereof.

7. The intelligent system according to claim 1, wherein the ideal knee movement includes ideal knee angle, knee velocity, angular velocity of knee, angular acceleration of knee or muscle activity in spatiotemporal patterns.

8. The intelligent system according to claim 1, wherein the analyzer is configured to compare the predicted knee movement and the ideal knee movement, which generates an error rate.

9. The intelligent system according to claim 8, wherein the error rate meets a formula as follows:
$$error rate = \frac{current knee movement - ideal knee movement}{ideal knee movement} .$$

10. The intelligent system according to claim 8, wherein the intelligent system further includes at least one parameter convertor, which is configured to adjust parameters regulating the functional unit according to the error rate; and wherein the parameter convertor is communicatively connected between the analyzer and the functional unit.

11. The intelligent system according to claim 1, wherein the intelligent system further includes at least one parameter convertor, which is configured to adjust parameters regulating the functional unit according to comparison results from the analyzer; and wherein the parameter convertor is communicatively connected between the analyzer and the functional unit.

12. The intelligent system according to claim 11, wherein the parameter convertor includes a vibration, infrared stimulation, laser, thermal or electrical stimulation parameter convertor.

13. The intelligent system according to claim 11, wherein the parameter convertor is configured to adjust at least one parameter of amplitude, frequency, frequency range, time duration, action sites, spatiotemporal pattern, infrared wavelength and temperature.

14. The intelligent system according to claim 11, wherein the parameter convertor is configured to adjust parameters through Deep Neural Network (DNN) model, a Convolutional Neural Network (CNN) model, Recurrent Neural Network (RNN) model or combination thereof.

15. The intelligent system according to claim 1, wherein the functional unit includes a vibration motor, an infrared instrument, a laser instrument, a heating instrument, a cooling instrument or an electrical stimulation instrument.

16. A use of an intelligent system, wherein the use includes purposes of health-care, rehabilitation, preventive medicine or medical use.
